(19) United States
(12) Patent Application Publication
(10) Pub. No.: US 2024/0342063 A1
(43) Pub. Date: Oct. 17, 2024

(54) AMORPHOUS CALCIUM PHOSPHATE GEL, PREPARATION METHOD AND USE THEREOF

(71) Applicant: Yunnan Baiyao Group Health Products Co., Ltd., Kunming (CN)

(72) Inventors: Lixian LI, Kunming (CN); Kegong NING, Kunming (CN); Qiuxia ZHANG, Kunming (CN); Ping LIU, Kunming (CN); Peng WU, Kunming (CN); Xiangye KONG, Kunming (CN); Ying CAI, Kunming (CN); Feng GAO, Kunming (CN)

(73) Assignee: Yunnan Baiyao Group Health Products Co., Ltd., Kunming (CN)

(21) Appl. No.: 18/320,218

(22) Filed: May 19, 2023

(30) Foreign Application Priority Data
Apr. 11, 2023 (CN) 202310382857.3

(51) Int. Cl. A61K 8/24; A61K 8/04; A61Q 11/00

(52) U.S. Cl. CPC A61K 8/24; A61K 8/042; A61Q 11/00; A61K 2800/92

(57) ABSTRACT

Disclosed is an amorphous calcium phosphate gel, as well as a preparation method and use thereof. The method comprises: adding aminomethyl propanol to a propylene glycol solution containing a calcium source to obtain a mixture; slowly adding a phosphoric acid solution to the mixture to obtain the amorphous calcium phosphate gel. The amorphous calcium phosphate gel as provided in the present disclosure has an excellent stability, which shows no obvious phase change after being left at room temperature for 12 months. The method avoids the use of toxic and harmful agents, which allows the production process to be safe and environmentally friendly, and also ensures the safety of the product. The method has a simple process, which allows the large-scale production of the product and provides a basis for the wide application of the amorphous calcium phosphate gel in oral care.

### CROSS-REFERENCE TO RELATED APPLICATIONS

This application claims priority to Chinese Patent Application No. 202310382857.3 with a filing date of Apr. 11, 2023. The content of the aforementioned application, including any intervening amendments thereto, is incorporated herein by reference.

### FIELD

The present disclosure relates to the technical field of oral care, in particular to an amorphous calcium phosphate gel, as well as a preparation method and use thereof.

### BACKGROUND

Dental tissues include three calcified hard tissues of enamel, dentin and cementum, and a soft tissue of dental pulp. Among them, the enamel is the hardest component of the dental tissues, covers the entire outer layer of the crown, and is the first line of the entire dental tissues against external damage. The enamel comprises inorganic matters of 96%-97%, organic matters of 0.4%-0.9% and water of 3.6%-2.1% on the total weight of the enamel, in which the inorganic matters mainly exist in a crystalline form of hydroxyapatite.

Due to long-term exposure to oral cavity, the dental tissues are immersed in saliva for a long time and invaded by dental plaque, which combined with long-term consumption of acidic food and drinks, results in that the enamel on the surface of teeth is acid-etched and demineralized. The acid-etching of the enamel is mainly resulted from the fact that hydroxyapatite crystals, the main component of the enamel, are in a dynamic equilibrium between dissolution and redeposition when immersed in saliva or drinks, where the rate of dissolution depends on the pH of the contact solution as well as the concentration of calcium and phosphorus ions in the contact solution. When the contact solution has a pH value below 5.5 and does not contain sufficient calcium and phosphate ions, the hydroxyapatite in the enamel tends to dissolve, which easily leads to demineralization of the enamel. When the contact solution is enriched with high concentrations of calcium and phosphate ions and has a pH of >5.5, the reaction equilibrium moves towards calcium phosphate deposition, which allows remineralization of the demineralized teeth. Continuous acid-etching and demineralization of the enamel is one of the main causes of tooth sensitivity and caries.

Amorphous calcium phosphate is rich in calcium ions and phosphate ions, and it can strengthen the enamel, prevent dental caries and resist tooth sensitivity when added to oral care products. At present, the amorphous calcium phosphate prepared by a sol-gel method suffers from poor stability, which leads to its limited application in oral care products.

### SUMMARY

To this end, the present disclosure provides an amorphous calcium phosphate gel, as well as a preparation method and use thereof.

In order to achieve the above objects, the present disclosure provides the following technical solutions.

According to a first aspect of the present disclosure, there is provided a method for preparing an amorphous calcium phosphate gel, which comprises: adding aminomethyl propanol to a propylene glycol solution containing a calcium source to obtain a mixture; and slowly adding a phosphoric acid solution to the mixture to obtain the amorphous calcium phosphate gel.

In the present disclosure, the amorphous calcium phosphate gel is prepared by using an anhydrous system. The applicant found through extensive researches that, the amorphous calcium phosphate gel, which is prepared by adding aminomethyl propanol (C₄H₁₁NO, CAS No. 124-68-5) as a phase stabilizer to a propylene glycol solution containing a calcium source, can undergo no phase change for a longer period of time and has an excellent stability. Meanwhile, the agents used in this method are safe, avoiding the harm to human body to the maximum extent.

Further, the mass ratio of the aminomethyl propanol to the propylene glycol solution containing the calcium source is 1:20˜1:2. It was found that the amount of the aminomethyl propanol as the phase stabilizer significantly affects the stability of the product. When the amount of the aminomethyl propanol is low, the prepared amorphous calcium phosphate gel is prone to phase transformation to crystalline calcium phosphate, and when the amount of the aminomethyl propanol is high, the prepared amorphous calcium phosphate gel has an increased pH value, which is detrimental to its wide application in oral care products. With the above mass ratio of the aminomethyl propanol to the propylene glycol solution containing the calcium source, the stability of the amorphous calcium phosphate gel can be improved, and it is also favorable to expand its application in oral care products.

Further, the molar concentration of phosphoric acid in the phosphoric acid solution is 0.03˜2 mol/L, and the mass ratio of the phosphoric acid solution to the mixture is 1:5˜4:5. It was found that when the molar concentration of phosphoric acid is too low or too high, the prepared amorphous calcium phosphate tends to transform to a crystalline state. Under the above conditions, it is more favorable to improve the stability of the amorphous calcium phosphate gel.

Further, the phosphoric acid solution is a mixed solution of phosphoric acid and an organic solvent, and the organic solvent is propylene glycol and/or glycerin, preferably propylene glycol.

Further, the molar concentration of calcium ions in the propylene glycol solution containing the calcium source is 0.02˜1 mol/L, and the calcium source is calcium citrate, calcium acetate, calcium gluconate, calcium hydride, calcium fluoride, calcium chloride, or a calcium chloride hydrate. Among them, the calcium chloride hydrate is calcium chloride dihydrate, calcium chloride tetrahydrate or calcium chloride hexahydrate. As the calcium chloride and hydrate thereof are more readily available and have better solubility in propylene glycol, the calcium source is preferably calcium chloride or a calcium chloride hydrate.

Further, the phosphoric acid solution is added in an addition rate of 20˜500 ml/min. It was found that the addition rate thereof affects the crystalline morphology of calcium phosphate. When the addition rate is too slow, the generated amorphous calcium phosphate is not easily dispersed, resulting in a high local concentration for phase transformation, and when the addition rate is too fast, no amorphous calcium phosphate gel can be obtained.

Further, the propylene glycol solution containing the calcium source is prepared by stirring the calcium source and propylene glycol until dissolved at a temperature of 60˜90° C., and then cooling to room temperature to obtain a clear liquid.

According to a second aspect of the present disclosure, there is provided an amorphous calcium phosphate gel prepared by any one of the above-mentioned methods.

According to a third aspect of the present disclosure, there is provided use of the above-mentioned amorphous calcium phosphate gel in an oral care product, wherein the oral care product is an anhydrous oral care product. The anhydrous oral care product is preferably a gel or a toothpaste.

Further, the mass percentage of the amorphous calcium phosphate gel in the oral care product is 10%˜50%.

It will be appreciated that the anhydrous oral care product further comprises an adjuvant commonly used in oral care, such as a thickening agent, a solvent, a sweetener, an abrasive, a surfactant and an essence, etc. Among them, the thickening agent can be carbomer or xanthan gum, etc.; the solvent can be polyethylene glycol, glycerin or propylene glycol, etc.; the sweetener can be xylitol, sucralose or sodium saccharin, etc.; the abrasive can be hydrated silica, calcium hydrogen phosphate or calcium carbonate, etc.; the surfactant can be sodium methyl taurate, sodium lauryl sulfate or cocoyl propyl betaine, etc.; and the essence is a blended flavor which is formulated using several or even dozens of flavors in a certain ratio to have a certain aroma or fragrance and have a certain purpose.

The anhydrous oral care product can be prepared according to the conventional methods in the art. As an example, when the anhydrous oral care product is a gel, it is prepared by a method including:
(1) placing a solvent in an emulsifying stirring pot and adding a thickening agent to be homogenized uniformly under stirring, then heating up to 50˜80° C. for 10˜30 min under stirring to allow the colloid to be fully swollen, and cooling down to below 30° C., to obtain a first mixture;
(2) adding a sweetener to the first mixture, and mixing uniformly under stirring at a low speed to obtain a second mixture;
(3) adding the amorphous calcium phosphate gel to the second mixture, and mixing uniformly under stirring at a low speed to obtain a third mixture; and
(4) adding an essence to the third mixture, mixing uniformly under stirring at a low speed, and then degassing to obtain the gel.

When the anhydrous oral care product is a toothpaste, it is prepared by a method including:
(1) placing a solvent in an emulsifying stirring pot and adding a thickening agent to be homogenized uniformly under stirring, then heating up to 50˜80° C. for 10˜30 min under stirring to allow the colloid to be fully swollen, and cooling down to below 30° C., to obtain a first mixture;
(2) adding a sweetener to the first mixture, and mixing uniformly under stirring at a low speed to obtain a second mixture;
(3) adding the amorphous calcium phosphate gel to the second mixture, and mixing uniformly under stirring at a low speed to obtain a third mixture;
(4) adding an abrasive to the third mixture, and mixing uniformly under stirring at a low speed to obtain a fourth mixture;
(5) adding an essence and/or a surfactant to the fourth mixture, mixing uniformly under stirring at a low speed, and then degassing to obtain the toothpaste.

The present disclosure has the following advantages.

The amorphous calcium phosphate gel as provided in the present disclosure has an excellent stability, which shows no obvious phase change after being left at room temperature for 12 months. The method for preparing the amorphous calcium phosphate gel avoids the use of toxic and harmful agents, which allows the production process to be safe and environmentally friendly, and also ensures the safety of the product. In addition, the method has a simple process, which allows the large-scale production of the product and provides a basis for the wide application of the amorphous calcium phosphate gel in oral care.

### BRIEF DESCRIPTION OF DRAWINGS

In order to further clearly illustrate the embodiments of the present disclosure or the technical solutions in the prior art, the drawings used in the description of the embodiments or the prior art will be briefly described below. It is apparent that the drawings in the following description are only exemplary, from which those of ordinary skill in the art can derive drawings of other embodiments without any creative work.

FIG. 1 is a transmission electron micrograph of the amorphous calcium phosphate gel prepared in Example 1 of the present disclosure which is left at room temperature for 1 week;

FIG. 2 is a transmission electron micrograph of the amorphous calcium phosphate gel prepared in Example 1 of the present disclosure which is left at room temperature for 3 months;

FIG. 3 is a transmission electron micrograph of the amorphous calcium phosphate gel prepared in Example 1 of the present disclosure which is left at room temperature for 12 months;

FIG. 4 is a X-ray diffraction pattern of the amorphous calcium phosphate gel prepared in Example 1 of the present disclosure;

FIG. 5 is scanning electron micrographs of a dental module before and after being treated with the oral care product prepared in Example 4 of the present disclosure: (a) before treatment, (b) after treatment.

### DETAILED DESCRIPTION

The embodiments of the present disclosure are illustrated by the specific examples hereinafter. Those of ordinary skill in the art can easily understand the other advantages and effects of the present disclosure from the contents disclosed in this specification. It is apparent that the described examples are only some rather than all of the examples of the present disclosure. Based on the examples in the present disclosure, any other examples obtained by those skilled in the art without any creative work are within the protection scope of the present disclosure.

### Example 1

This example provides a method for preparing an amorphous calcium phosphate gel.
(1) 3000 g of propylene glycol was placed in an emulsifying pot, and 300 g of calcium chloride dihydrate was added thereto. Calcium chloride dihydrate was completely dissolved in propylene glycol by stirring and simultaneously heating to 85° C. After cooling down to room temperature, 700 g aminomethyl propanol was added to the obtained clear liquid, and stirred uniformly to obtain a mixture;
(2) 180 g of phosphoric acid (85%) was dissolved in 1500 g of propylene glycol and mixed uniformly to obtain a phosphoric acid solution;
(3) The phosphoric acid solution of Step (2) was added to the mixture of Step (1) at a rate of 30 ml/min under a stirring condition of 20 rpm/min. After the addition was completed, an amorphous calcium phosphate gel was obtained.

### Example 2

This example provides a method for preparing an amorphous calcium phosphate gel.
(1) 3000 g of propylene glycol was placed in an emulsifying pot, and 28 g of calcium chloride was added thereto. Calcium chloride was completely dissolved in propylene glycol by stirring and simultaneously heating to 60° C. After cooling down to room temperature, 160 g aminomethyl propanol was added to the obtained clear liquid, and stirred uniformly to obtain a mixture;
(2) 20 g of phosphoric acid (85%) was dissolved in 1700 g of propylene glycol and stirred uniformly to obtain a phosphoric acid solution;
(3) The phosphoric acid solution of Step (2) was added to the mixture of Step (1) at a rate of 100 ml/min under a stirring condition of 25 rpm/min. After the addition was completed, an amorphous calcium phosphate gel was obtained.

### Example 3

This example provides a method for preparing an amorphous calcium phosphate gel.
(1) 100 g of calcium chloride, 250 g of calcium chloride dihydrate and 3000 g of propylene glycol were placed in an emulsifying pot. Calcium chloride and calcium chloride dihydrate were completely dissolved in propylene glycol by stirring and simultaneously heating to 60°° C. After cooling down to room temperature, 1200 g aminomethyl propanol was added to the obtained clear liquid, and stirred uniformly to obtain a mixture;
(2) 180 g of phosphoric acid (85%) was dissolved in 900 g of propylene glycol, and stirred uniformly to obtain a phosphoric acid solution;
(3) The phosphoric acid solution of Step (2) was added to the mixture of Step (1) at a rate of 50 ml/min under a stirring condition of 15 rpm/min. After the addition was completed, an amorphous calcium phosphate gel was obtained.

### Example 4

This example provides an oral care product (gel), in parts by weight, consisting of 45.5 parts of the amorphous calcium phosphate gel prepared in Example 1, 20 parts of polyethylene glycol, 30 parts of glycerin, 1.5 parts of carbomer, 2 parts of xylitol, and 1 part of an essence.

The oral care product was prepared by a method including the following steps.
(1) Polyethylene glycol was placed in an emulsifying stirring pot, carbomer was added to be homogenized uniformly under stirring, and glycerin was added. Thereafter, it was heated up to 60° C. for 15 min under stirring such that the colloid was fully swollen, and then cooled down to below 30° C. to obtain a first mixture;
(2) Xylitol was added to the first mixture to obtain a second mixture;
(3) The amorphous calcium phosphate gel was added to the second mixture, and mixed uniformly under stirring at a low speed to obtain a third mixture;
(4) The essence was added to the third mixture, mixed uniformly under stirring at a low speed, and degassed to obtain the gel.

### Example 5

This example provides an oral care product (toothpaste), in parts by weight, consisting of 30 parts of the amorphous calcium phosphate gel prepared in Example 3, 30 parts of polyethylene glycol, 1.5 parts of carbomer, 2 parts of xylitol, 20 parts of hydrated silica, 13.5 parts of calcium hydrogen phosphate, 1 part of an essence, and 2 parts of sodium methyl taurate.

The oral care product was prepared by a method including the following steps.
(1) Polyethylene glycol was placed in an emulsifying stirring pot, and carbomer was added to be homogenized uniformly under stirring. Thereafter, it was heated up to 60° C. for 15 min under stirring such that the colloid was fully swollen, and then cooled down to below 30° C. to obtain a first mixture;
(2) Xylitol was added to the first mixture to obtain a second mixture;
(3) The amorphous calcium phosphate gel was added to the second mixture, and mixed uniformly under stirring at a low speed to obtain a third mixture;
(4) Hydrated silica and calcium hydrogen phosphate were added to the third mixture, and mixed uniformly under stirring at a low speed to obtain a fourth mixture;
(5) The essence and sodium methyl taurate were added to the fourth mixture, mixed uniformly under stirring at a low speed, and degassed to obtain the toothpaste.

### Comparative Example 1

This comparative example provides a method for preparing an amorphous calcium phosphate gel, which was the same as the method of Example 1 except that aminomethyl propanol was not used.

### Test Examples

### 1. Transmission Electron Microscopy

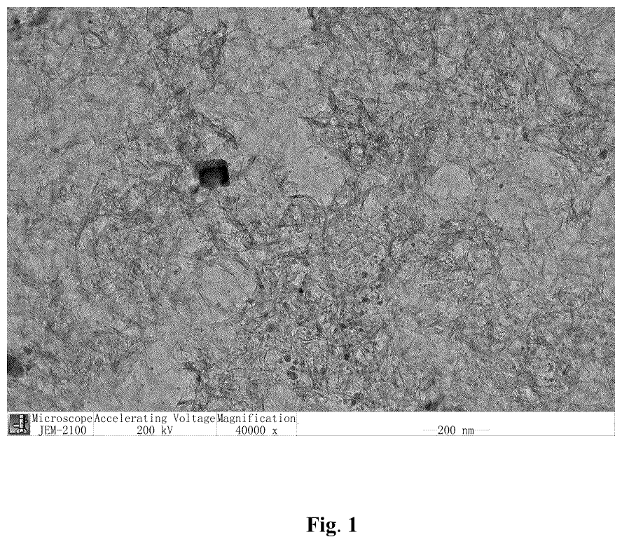

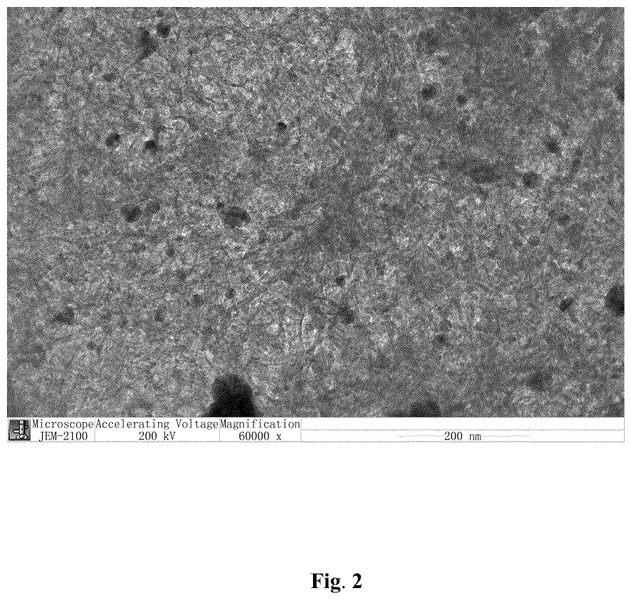

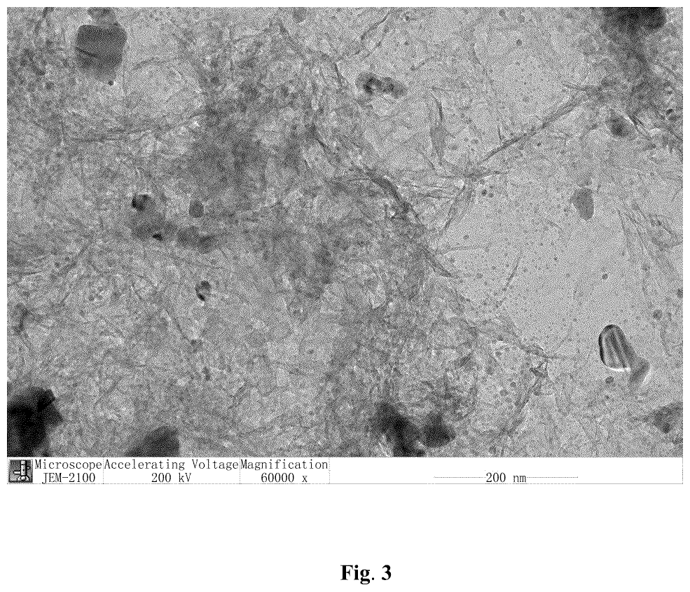

The sample of the amorphous calcium phosphate gel prepared in Example 1 was observed with a transmission electron microscope after it was left at room temperature (25±1° C.) for 1 week. As shown in FIG. 1, agglomerated micelles were observed. After the above sample was left under the same condition for 3 months and 12 months, the transmission electron micrographs (FIGS. 2-3) showed that they were still obvious agglomerated micelles and did not show significant aggregation, growth and crystallization. For the samples of the amorphous calcium phosphate gels prepared in Examples 2-3, after they were left at room temperature for 1 week, 3 months, and 12 months, the results thereof observed with the transmission electron microscope were not significantly different from those in Example 1.

The sample of the amorphous calcium phosphate prepared in Comparative Example 1 failed to form a stable gel state, and was transformed into a crystalline state after it was left at room temperature for 20 min.

The above results show that the amorphous calcium phosphate gel as provided in the present disclosure has an excellent stability.

### 2. X-Ray Diffraction

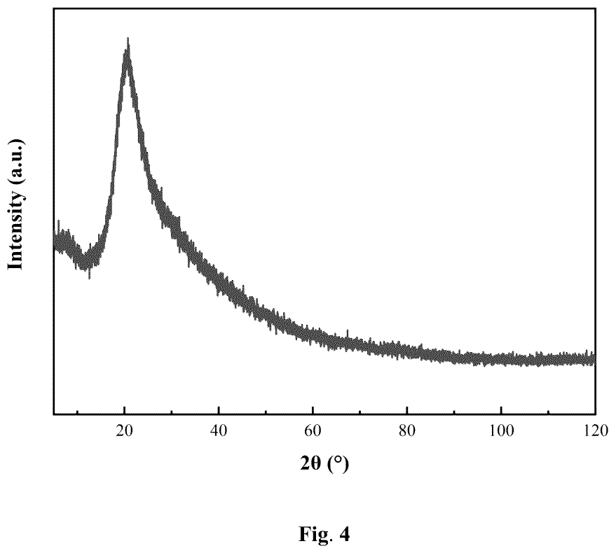

The amorphous calcium phosphate gel prepared in Example 1 was analyzed by X-ray diffraction. As shown in FIG. 4, no crystal peak was observed. The X-ray diffraction patterns of the amorphous calcium phosphate gel samples prepared in Examples 2-3 were not significantly different from that in Example 1.

The above results show that the amorphous calcium phosphate gel as provided in the present disclosure comprises no crystalline phase and is a pure amorphous phase.

### 3. Blocking of Dentin Pores

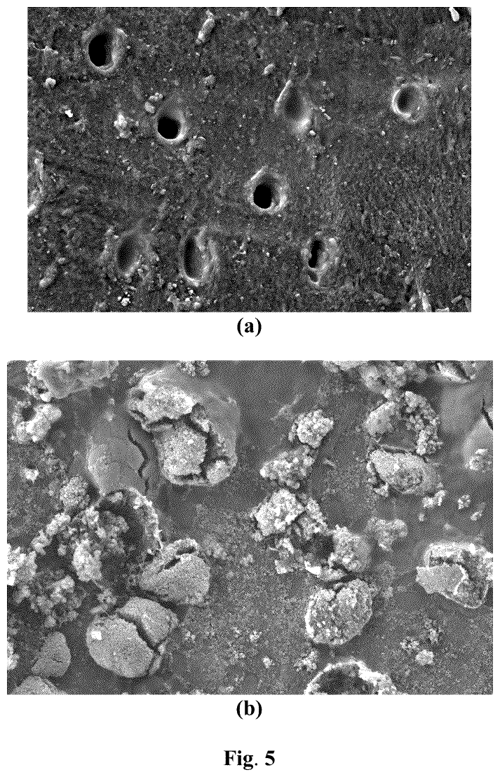

1 gram of the oral care product prepared in Example 4 was applied to a dental module having exposed dentin pores due to phosphoric acid etching, and left for 1 min. Thereafter, the sample remaining on the surface of the dental module was washed away. The dental module was dried and then observed by a scanning electron microscope. As shown in FIG. 5, the dentin pores could be effectively filled after treatment.

The above results show that the oral care product as provided in the present disclosure can effectively block the dentinal pores, thereby alleviating the tooth sensitivity due to the exposure of the dentinal pores.

Although the present disclosure has been described in detail above with general descriptions and specific embodiments, it is obvious to those skilled in the art to make some modifications or improvements on the basis of the present disclosure, which is apparent to those skilled in the art. Therefore, these modifications or improvements made without departing from the spirit of the present disclosure belong to the protection scope of the present disclosure.

What is claimed is:

1. A method for preparing an amorphous calcium phosphate gel, comprising:
adding aminomethyl propanol to a propylene glycol solution containing a calcium source to obtain a mixture;
slowly adding a phosphoric acid solution to the mixture to obtain the amorphous calcium phosphate gel.

2. The method according to claim 1, wherein,
a mass ratio of the aminomethyl propanol to the propylene glycol solution containing the calcium source is in a range of 1:20˜1:2.

3. The method according to claim 1, wherein,
a molar concentration of phosphoric acid in the phosphoric acid solution is in a range of 0.03˜2 mol/L, and a mass ratio of the phosphoric acid solution to the mixture is in a range of 1:5˜4:5.

4. The method according to claim 1, wherein,
the phosphoric acid solution is a mixed solution of phosphoric acid and an organic solvent, and the organic solvent is propylene glycol and/or glycerin.

5. The method according to claim 1, wherein,
a molar concentration of calcium ions in the propylene glycol solution containing the calcium source is in a range of 0.02˜1 mol/L, and the calcium source is calcium citrate, calcium acetate, calcium gluconate, calcium hydride, calcium fluoride, calcium chloride, or a calcium chloride hydrate.

6. The method according to claim 1, wherein the phosphoric acid solution is added at a speed of 20˜500 ml/min.

7. The method according to claim 1, wherein the propylene glycol solution containing the calcium source is prepared by stirring the calcium source and the propylene glycol until dissolved at a temperature of 60˜90° C., and then cooling to room temperature to obtain a clear liquid.

8. An amorphous calcium phosphate gel prepared by the method of claim 1.

9. Use of the amorphous calcium phosphate gel of claim 8 in an oral care product, wherein the oral care product is an anhydrous oral care product.

10. The use according to claim 9, wherein a mass percentage of the amorphous calcium phosphate gel in the oral care product is in a range of 10%˜50%.
